(19) United States
(12) Patent Application Publication
(10) Pub. No.: US 2015/0032020 A1
(43) Pub. Date: Jan. 29, 2015

(54) INHALATION SUPPORT APPARATUS AND METHOD FOR INHALATION SUPPORT

(71) Applicant: Dieter Kirsch, Bad Langensalza (DE)

(72) Inventors: Dieter Kirsch, Bad Langensalza (DE); Anja Kirsch, Bad Langensalza (DE)

(21) Appl. No.: 14/373,208

(22) PCT Filed: Nov. 9, 2012
(86) PCT No.: PCT/EP2012/072270

§ 371 Date: Jul. 18, 2014

(30) Foreign Application Priority Data
Jan. 20, 2012 (DE) 10 2012 200 815.2

(51) Int. Cl. A61M 16/00; A61B 5/00; A61M 16/14; A61B 5/097; A61M 16/04; A61M 16/10; A61M 16/08

(52) U.S. Cl. CPC A61M 16/0003; A61M 16/0051; A61M 16/105; A61B 5/4806; A61M 16/0858; A61B 5/097; A61B 5/4839; A61M 16/0488; A61M 16/14; A61M 2016/0027; A61M 2205/50; A61M 2205/35; A61M 2202/064
USPC 600/543; 128/203.14; 600/529

(57) ABSTRACT

The invention relates to an inhalation support apparatus and the use thereof and to a method for supporting the inhalation of an inhalation mixture that is to be inhaled. The invention further relates to a device for the control of pressure changes, their use in the medical and non-medical field and a method for detecting respiratory problems.

### FIELD OF THE INVENTION

The invention relates to an inhalation support apparatus for inhalation devices that have a mouthpiece and an inhalation mixture generator for supplying an inhalation mixture to be inhaled. The invention also relates to a method for supporting the inhalation of an inhalation mixture that is to be inhaled by means of an inhalation apparatus. The invention further relates to an apparatus for detecting pressure changes, to the use thereof in medical and non-medical fields, and to a method for identifying respiratory problems.

### BRIEF DISCLOSURE OF THE INVENTION

Inhalation is an effective method by which, for example in respiratory tract diseases, it is possible to bring active substances directly into the affected airways. The active substance is inhaled as a constituent part of an inhalation mixture.

A distinction is made between nebulizer or aerosol inhalation and dry inhalation. An aerosol of the active substance is generally used, or a mixture of vapor of a solvent (e.g. water) for the active substance and respiratory air. In dry inhalation, powdered active substances and additives are inhaled. The inhalation support apparatus according to the invention is suitable for use both in aerosol inhalation and also in dry inhalation.

Generally speaking, inhalation is especially effective if the inhaling person inhales and exhales uniformly and sufficiently deeply. A problem that often arises for the inhaling person is that of maintaining an appropriate breathing pattern or a suitable breathing rhythm. Inhalation can also be especially problematic for a child, for whom inhalation is often a tedious and unwelcome procedure.

The object of the invention is to support an inhaling person during inhalation or to encourage an effective inhalation process or inhalation rhythm.

This object is achieved by an inhalation support apparatus as claimed in claim 1, which is provided for use with inhalation devices that have a mouthpiece and an inhalation mixture generator for supplying an inhalation mixture to be inhaled. The inhalation support apparatus has a pressure sensor adapter, which can be arranged between the inhalation mixture generator and the mouthpiece of the inhalation device, wherein pressure changes of the inhalation mixture can be detected by the pressure sensor adapter. A control device is also provided, with which an entertainment program running on a data processing installation (not part of the inhalation support apparatus) is controlled as a function of the pressure changes detected by means of the pressure sensor adapter.

### BRIEF DESCRIPTION OF THE DRAWINGS

FIG. 1 shows an inhalation device;

FIG. 2 shows the inhalation device from FIG. 1 in a disassembled state;

FIG. 3 shows an inhalation support apparatus according to the invention;

FIG. 4 shows a detail of the inhalation support apparatus from FIG. 3;

FIG. 5 shows a further detail of the apparatus according to FIG. 3;

FIG. 6 shows an inhalation device with an inhalation support apparatus according to the invention;

FIG. 7 shows an apparatus for monitoring pressure changes.

### DETAILED DESCRIPTION OF THE INVENTION

The apparatus according to the invention serves to support the inhalation process in conventional inhalation devices. In such inhalation devices, the inhalation mixture to be inhaled flows generally from the inhalation mixture generator through an inhalation channel and through the mouthpiece via which a person can breathe in the inhalation mixture. The apparatus according to the invention can also easily be retrofitted in such an inhalation device, since these can often be disassembled for cleaning purposes. In addition, data processing installations (PCs, games consoles or the like, and cell phones) are already present nowadays in many households, for which reason the apparatus according to the invention can be used without any problem.

A basic distinction is made between three different inhalation systems: dosing aerosols (DA), powder inhalers, and electrical nebulizer systems. The inhalation support apparatus according to the invention is in principle suitable for use with all three inhalation systems.

In a preferred embodiment, the apparatus according to the invention is used together with a dosing aerosol or an electrical nebulizer system. In a nebulizer, the inhalation solution is sprayed through a nozzle either by ultrasound or by means of compressed air. An embodiment is particularly preferred in which the dosing aerosol is a propellant gas aerosol. An embodiment is most preferred in which the propellant gas aerosol additionally has a spacer.

In another preferred embodiment, the apparatus according to the invention is used together with a powder inhaler.

When the apparatus is operated together with an inhalation device and a data processing installation, the data processing installation is made accessible to the inhaling person. An entertainment program runs on the data processing installation. Since the entertainment program is controlled by the pressure changes of the inhalation mixture, the inhaling person is able, by controlled inhalation and exhalation, to influence the entertainment program via the pressure changes caused in the inhalation mixture. This is conceivable particularly with an entertainment program in the manner of a computer game in which certain actions in the game can be controlled via the detected pressure changes. In this way, the person can be supported in the inhalation. In particular, by means of a suitable configuration of the entertainment program, for example by means of stimuli generated at certain time intervals for influencing a game action for example, an inhaling person can be specifically incited to inhale and exhale. In this way, the inhalation process is supported almost in the manner of a game.

In the present context, the stated detection of pressure changes by means of the pressure sensor adapter does not necessarily already denote a conversion of the pressure change into electrical measurement signals, as may happen with a pressure transducer or a pressure sensor, for example. Instead, it is advantageous if the pressure acting (instantaneously) in the inhalation mixture (in particular the pressure prevailing in the mouthpiece or in an inhalation channel for passage of the inhalation mixture from the inhalation mixture generator to the mouthpiece) or the change in this pressure is conveyed to a pressure transducer via a communicating pressure line, i.e. a pressure connection. In particular, the detected pressure changes are fed to the control device in such a way that the pressure changes also act in the control device (or on a measurement transducer provided in/on the control device). In this case, a conversion to electrical measurement signals takes place in the control device, arranged in particular at a distance from the pressure sensor adapter.

The control device preferably comprises a conventional processor, the pressure transducer, and software for controlling the components and signal conversion. The pressure transducer is preferably a pressure sensor. A differential pressure sensor which has a high degree of sensitivity is particularly preferred.

The detected pressure changes can preferably be converted by means of the control device into control signals for controlling the entertainment program running on the data processing installation. For this purpose, the control device interacts with the data processing installation via a communication link, for example a serial or parallel interface, USB connection, mini USB connection or WLAN connection or Bluetooth connection.

In a particularly preferred embodiment, the pressure sensor adapter can be arranged releasably between the inhalation mixture generator and the mouthpiece, in particular being able to be plugged between them.

It is thus possible to arrange the apparatus according to the invention in a conventional inhalation device only when so required. However, the inhalation support apparatus can also be separated again from the inhaler, thereby permitting convenient and thorough cleaning, in particular also of the connection sites to the mouthpiece and to the inhalation mixture generator.

It is also advantageous if the pressure sensor adapter has an inlet plug section for joining to an associated plug section of the inhalation mixture generator, and also an outlet plug section for joining to an associated plug section of the mouthpiece. In conventional inhalation devices, the mouthpiece is often attachable, for example in order to permit cleaning in a simple way. The inlet plug section and the outlet plug section are preferably adapted to the corresponding plug sections of conventional inhalation devices.

For the further configuration of the apparatus according to the invention, provision is made that the pressure sensor adapter has a through-flow channel for passage of the inhalation mixture, in particular from the inhalation mixture generator to the mouthpiece, and a pressure sensor port communicating in terms of pressure with the through-flow channel. The pressure sensor port thus interacts with the through-flow channel in such a way that the pressure of the inhalation mixture in the through-flow channel can be transmitted or forwarded via the pressure sensor port, or in such a way that the pressure of the inhalation mixture also prevails in the pressure sensor port, or in a pressure sensor space connected thereto, or in a pressure sensor line connected thereto.

However, it is also conceivable that the pressure sensor adapter has a pressure transducer which interacts with the through-flow channel in such a way that the pressure in the through-flow channel can be measured.

It is particularly advantageous if the pressure sensor port opens into the through-flow channel. The through-flow channel has in particular an effective cross-sectional area (through-flow cross section) for the flow of the inhalation mixture, and the pressure sensor port has an effective cross-sectional area (pressure sensor cross section), wherein the pressure sensor Cross section is much smaller than the through-flow cross section, and in particular the ratio of pressure sensor cross section to through-flow cross section is in the range of 1:100 to 1:10. It is thus possible to avoid a situation where the pressure sensor port causes a noticeable drop in pressure in the through-flow channel.

An embodiment is preferred in which the ratio of pressure sensor cross section to through-flow cross section has a ratio of 1:100, 1:90, 1:80, 1:70, 1:60, 1:50, 1:40, 1:30, 1:20 or 1:10. An embodiment is particularly preferred in which the ratio of pressure sensor cross section to through-flow cross section has a ratio of 1:100, 1:90, 1:80, 1:70, 1:60 or 1:50. An embodiment is likewise preferred in which the ratio of pressure sensor cross section to through-flow cross is in the range of 1:100 to 1:50. An embodiment is furthermore preferred in which the ratio of pressure sensor cross section to through-flow cross section has a ratio of 1:100, 1:90, 1:80 or 1:70. An embodiment is furthermore preferred in which the ratio of pressure sensor cross section to through-flow cross section is in the range of 1:100 to 1:70. Most preferred is an embodiment in which the ratio of pressure sensor cross section to through-flow cross section has a ratio of 1:100 or 1:90. An embodiment is likewise preferred in which the ratio of pressure sensor cross section to through-flow cross section is in the range of 1:100 to 1:90.

The pressure sensor port opens out in particular into an inner wall of the through-flow channel conveying the inhalation mixture.

A further advantageous embodiment of the apparatus is one in which the pressure sensor adapter is pressure-connected to the control device via a flexible pressure sensor line for forwarding the detected pressure or the detected pressure changes. This permits a more comfortable use of the apparatus according to the invention during the inhalation and simultaneous influence over a computer program on the attached data processing installation.

The pressure sensor line is thus arranged between and connects the pressure sensor adapter and the control device. In particular, the pressure sensor line is pressure-connected to the pressure sensor port in such a way that the detected pressure is forwarded through the pressure sensor line. The control device has in particular a pressure port via which pressure changes can be fed to the control device. The pressure sensor line can be pressure-connected to this pressure port.

For further configuration, a filter element is provided in the pressure sensor line, which filter element in particular traps liquid droplets contained in the inhalation mixture and/or impurities, for example pathogenic microorganisms. The filter element is arranged in particular in the pressure sensor line between the pressure sensor adapter and the control device. It is thus possible to avoid liquid or impurities (for example from the inhalation mixture or from the exhaled air) getting into the control device and there leading to contamination or damage. It is also conceivable that the filter element is arranged in or on the pressure sensor adapter or in or on the control device.

Suitable filter elements have a filter capsule, which encloses a filter material, and also an inlet and an outlet, in such a way that a gaseous medium can flow through the inlet to the filter medium and, after passing through the filter medium, can flow out from the outlet.

Examples of filters suitable for the retention of microorganisms are conventional sterile filters, such as bacterial filters or viral filters, which are constructed as described above.

Suitable filter media for the retention of moisture or liquid droplets are moisture adsorbers, for example commercially available silica gels or zeolites.

Further suitable filter media are membranes with a very narrow pore width, which are able to retain both microorganisms and also liquid droplets. Such membranes are preferably made of Teflon.

In the apparatus according to the invention, it is also possible for two or more filter elements to be arranged in succession, for example a bacterial filter and a filter element that contains an adsorber material.

In a particularly preferred embodiment of the inhalation support apparatus, the pressure sensor adapter is free of electronics, that is to say is designed without electronic components or free of current-carrying or voltage-carrying components.

In particular, in this embodiment, the pressure sensor adapter is free of measurement electronics. The required pressure transducer is likewise not arranged in the pressure sensor adapter. These electronic-free configurations of the pressure sensor adapter have the advantage that cleaning with liquid is also easily possible, and damage to the measurement electronics, for example by liquid, can be avoided. For example, the pressure sensor adapter can be cleaned without any problem under running water. Inhalation devices can often be disassembled for cleaning, in particular the mouthpiece can be separated from the inhalation mixture generator. Those parts that come into contact with moisture or with inhalation mixture or with respiratory air can then be cleaned individually and thoroughly.

In order to convert the detected pressure changes into electrical measurement signals, the control device preferably has a pressure transducer. This pressure transducer is in particular not provided in or on the pressure sensor adapter but instead in or on the control device. In particular, the control device has a pressure port via which the detected pressure or the detected pressure changes can be fed to the pressure transducer of the control device.

An advantageous embodiment of the inhalation support apparatus is one in which the control device has a housing, wherein the housing has a plug connector connected to the housing for the purpose of establishing a communication link with the data processing installation. The plug connector is preferably rigidly connected to the housing. This plug connector can in particular be designed as a USB plug and can be integrated in the housing of the control device or rigidly connected to the housing of the control device, in such a way that the housing along with the USB plug can be plugged into a USB socket of the data processing installation. This allows the inhalation support apparatus to be conveniently attached for operation to the data processing installation.

The pressure transducer, in particular along with all the necessary control electronics, is preferably enclosed in the housing of the control device. The housing preferably has a pressure inlet section, to which the pressure sensor line can be connected for example, in order to convey the pressure changes to the control device or to a pressure transducer likewise accommodated in the housing.

In a particularly preferred embodiment of the apparatus according to the invention, the entertainment program is stored or can be stored in the control device. For this purpose, the control device can have a memory, for example a flash memory. For operation, the control device can be connected to the data processing installation, and the entertainment program can then be executed on the data processing installation. The inhalation support apparatus designed in this way can thus be easily operated in the manner of a plug and play appliance.

However, it may also be advantageous if the entertainment program is stored in the data processing installation and also runs there. Thus, for example, different entertainment programs, from which the inhaling person can choose, can be made available for the apparatus according to the invention. This therefore permits varied inhalation support.

The object is also achieved by an inhalation apparatus with an inhalation mixture generator for supplying an inhalation mixture to be inhaled, and with a mouthpiece through which the inhalation mixture can be inhaled, wherein an inhalation support apparatus of the kind described above is provided whose pressure sensor adapter is arranged between the inhalation mixture generator and the mouthpiece. The pressure sensor adapter is in particular arranged in such a way that, during the operation of the inhalation apparatus, the inhalation mixture flows through the pressure sensor adapter.

The object mentioned at the outset is also achieved by a method for supporting the inhalation of an inhalation mixture that is to be inhaled by means of an inhalation apparatus. This method involves detecting a pressure change of the inhalation mixture that is to be inhaled, converting the pressure change into control signals for controlling a data processing installation, and controlling an entertainment program running on the data processing installation by means of the control signals and as a function of the pressure change detected. After the pressure change has been detected, this pressure change is fed in particular to a control device for conversion into control signals.

As has already been described above, the inhaling person can thus be made to initiate pressure changes in the inhalation mixture, by means of suitably controlled inhalation and exhalation, and thereby influence the entertainment program. It is conceivable in particular that, by emitting suitable stimulus signals (for example images, sounds, etc.), the entertainment program prompts the person to carry out a specific game action at predeterminable successive time intervals. In the entertainment program, this game action can then be brought about for example by specific inhalation and exhalation. In this way, for example, it is possible to predefine an inhalation rhythm that is desirable or particularly expedient for therapeutic purposes.

The inhalation apparatus according to the invention is particularly suitable for use in the treatment and/or alleviation of diseases.

Accordingly, a further embodiment of the invention also concerns the inhalation support apparatus according to the invention for use in the treatment and/or alleviation of diseases.

A further embodiment of the invention concerns the use of the inhalation support apparatus according to the invention for the treatment and/or alleviation of diseases.

The invention further relates to a method for treatment and/or alleviation of diseases, wherein the inhalation support apparatus according to the invention is used for the treatment of the patients.

The apparatus according to the invention is especially suitable for the treatment and/or alleviation of diseases that are treatable by means of inhalation. With the aid of the inhalation support apparatus according to the invention, it is possible to achieve a particularly uniform and continuous dosing of the medicaments required for the disease in question.

Examples of diseases that are treatable by inhalation are diseases of the bronchi and of the nasopharyngeal space, chronic or acute respiratory tract diseases, and severe viral or bacterial infections of the airways.

In a preferred embodiment, the inhalation support apparatus according to the invention is used for example in the treatment of bronchitis, asthma, celiac disease, COPD (chronic obstructive pulmonary disease), allergic rhinitis, pneumonia, fungal diseases of the lungs, pulmonary tuberculosis, or severe acute respiratory syndrome (SARS).

In another preferred embodiment, the inhalation support apparatus according to the invention is used for the alleviation of symptoms of metabolic disorders, in particular of metabolic disorders caused by a genetic defect.

An example of one such by a genetic is cystic fibrosis, also called mucoviscidosis. Cystic fibrosis is caused by a genetic defect on human chromosome 7. Symptoms of the disease are persistent coughing, possibly frequent pneumonia, shortness of breath, digestive problems with abdominal pain, poor growth, and poor weight gain. Despite considerable efforts in medical and pharmaceutical research, cystic fibrosis is as yet incurable. The treatment of this disease is therefore limited to continuously improving the conventional treatment methods and to managing frequent concomitant diseases such as infections of the airways or pneumonia.

Inhalation is of increasing importance in the therapeutic treatment of patients with cystic fibrosis. While inhalation therapy at the outset was aimed mainly at loosening the viscous mucus in the airways, so that the affected persons can more easily cough the mucus up, inhalation therapy is nowadays being used increasingly to administer medicaments. Medicaments that act on the lungs can thus arrive directly at the target site. In accordance with the different goals of treatment, there are also different inhalation substances that patients with cystic fibrosis can inhale. The most important inhalation substances are mucolytic substances, broncho-dilating substances, anti-inflammatory substances, and antibiotics for the treatment of bacterial infections of the lungs.

A particularly preferred embodiment of the invention therefore concerns the use of the inhalation support apparatus in methods for the treatment of cystic fibrosis. The use of the apparatus according to the invention is particularly advantageous in the treatment of cystic fibrosis when the inhaling person is a child and when the child is assisted by the apparatus during inhalation or is prompted to an effective inhalation process or inhalation rhythm.

Medicaments that are administered by means of inhalation have also been recently developed for the treatment of other diseases. One example is the administration of inhalable insulin for the treatment of type 1 and type 2 diabetes. A further preferred embodiment of the invention therefore concerns the use of the inhalation support apparatus in methods for the treatment of type 1 and type 2 diabetes.

The control device has a pressure transducer, as has been described above, and is in principle suitable for converting the pressure changes, delivered via a pressure sensor line, into electrical measurement signals. A further embodiment of the invention thus concerns an apparatus for monitoring pressure changes. This apparatus for monitoring pressure changes comprises a control device, which has a pressure transducer, a pressure sensor hose and, optionally, a filter element, wherein the control device has a housing with a pressure port. The control device can optionally have a plug connector. This is especially the case when the connection to a data processing installation is produced not by a wireless connection but instead by a plug-type connection.

An apparatus set up in this way can be used wherever pressure changes occur and are able to be detected. The apparatus can be used both in medical and in non-medical fields.

An appliance on which the pressure changes are intended to be measured can preferably be pressure-connected, with the aid of a pressure sensor hose, to the control device for the purpose of delivering the detected pressure changes.

By means of a suitable computer program stored on an attached data processing installation or in the control device itself, the electrical measurement signals generated in the control device can be used to generate measurement data that can be used for the diagnosis of respiratory problems.

A further embodiment of the invention thus concerns an apparatus for monitoring pressure changes during use in the diagnosis of respiratory problems.

The invention further relates to the use of the apparatus for monitoring pressure changes for the diagnosis of respiratory problems.

The invention further relates to a method for identifying respiratory problems, wherein the apparatus according to the invention is used for monitoring pressure changes.

A method according to the invention for identifying respiratory problems comprises the following steps for example:
attaching the apparatus for detection of pressure changes to a data processing installation;
attaching the apparatus according to the invention, by means of a pressure sensor hose, to an appliance for monitoring the respiration of a patient, in such a way that the control device is pressure-connected to the appliance for monitoring the respiration;
detecting pressure changes and generating electrical measurement signals in the control device;
converting the electrical measurement signals from the control device into measurement data by means of a suitable computer program stored on the data processing installation;
storing the measurement data on the data processing installation;
evaluating the measurement data, in particular comparing the measurement data obtained from a patient against the data from a healthy subject who has normal respiration;
determining divergences or impairments of the respiration of the patient in relation to the normal respiration of a healthy subject;
diagnosing the nature of the respiratory problems.

In this method, a suitable data processing installation can be any conventional computer, for example a desktop PC, a laptop PC or a tablet PC, but also any data processing installation integrated in a medical apparatus or diagnostic appliance. A cell phone, for example a smart phone, is likewise suitable as data processing installation.

In the method for detecting respiratory problems, a medical apparatus is preferably used in which a corresponding data processing installation is integrated and which, after evaluation of the collected measurement data, can be used for the immediate treatment of the respiratory problems. A method is particularly preferred in which the evaluation of the measurement data and the treatment of the respiratory problems take place automatically.

The apparatus according to the invention for monitoring pressure changes and the method for detecting respiratory problems can be used equally in veterinary medicine and human medicine.

The apparatus according to the invention for monitoring pressure changes is particularly suitable for use in diagnostic methods in sleep medicine. In a preferred embodiment, the apparatus according to the invention is used for the diagnosis of sleep disturbances, for example snoring. The apparatus according to the invention is particularly preferably used for the diagnosis of sleep apnea syndrome. Sleep apnea syndrome is almost always associated with intense snoring. There are pauses in breathing. These pauses can occur many times during the night. As a result, the snorer never attains the deep sleep stage that is needed for a restful sleep. On the next day, he is tired and exhausted. The pauses in breathing resume the following night. The patient is then permanently exhausted.

Sleep disturbances such as sleep apnea syndrome are treated by the sleep laboratory using a special sleep mask. The latter pumps air at a slight positive pressure into the airways of the patient. As a result, the patient is again able to have a full night's sleep without disturbance. The apparatus according to the invention for monitoring pressure changes can be attached to such a sleep mask and be used to detect the pauses in breathing. With the aid of the electrical measurement signals generated in the control device, control signals can be generated for controlling a computer program running on an attached data processing installation 62, as a result of which the air supply and the positive pressure in the sleep mask can be adapted to the requirements and optimally controlled.

In another preferred embodiment, the apparatus 70 for monitoring pressure changes is used in diagnostic methods for asthma, celiac disease and COPD.

Similarly, the apparatus according to the invention for monitoring pressure changes can be used to monitor respiration, for example in fitness tests, or to carry out lung function tests.

Other fields of use of the apparatus according to the invention for monitoring pressure changes include respiratory training for the elderly, or lung training, for example of the kind carried out in diving or parachuting. A further possible use of the apparatus according to the invention for detecting pressure changes is as a game. The apparatus is in this case pressure-connected in a suitable manner to a mouthpiece. In the operation of the apparatus together with a data processing installation, the data processing installation is made accessible to the person playing. An entertainment program runs on the data processing installation. Since the entertainment program is controlled by the pressure changes in the apparatus, the person playing is able, by controlled inhalation and exhalation, to influence the entertainment program via the pressure changes thereby caused. This is conceivable particularly with an entertainment program in the manner of a computer game in which certain actions in the game can be controlled via the detected pressure changes.

Further details and advantageous configurations of the invention are set forth in the following description, in which the embodiments of the invention shown in the figures are described and explained in more detail.

For the sake of clarity, the same reference signs are used below for identical or corresponding structural parts in all of the figures.

### 1. Inhalation Support Apparatus

FIG. 1 shows an inhalation device 10 which comprises a mouthpiece 12 and an inhalation mixture generator 14. The inhalation mixture generator 14 is used to supply an inhalation mixture that is to be inhaled (for example an aerosol of an in particular liquid active substance). For this purpose, the inhalation mixture generator 14 can have a liquid-receiving tank (not shown) for the active substance. In addition, a nebulizer (not shown) can be provided, by means of which the liquid active substance can be converted into an aerosol, for example by compressed air. For this purpose, a compressor for supplying the required compressed air for the nebulizer can be provided on the inhalation mixture generator 14, for example via a compressed air attachment (not shown).

The inhaling person can breathe the inhalation mixture in through the mouthpiece 12.

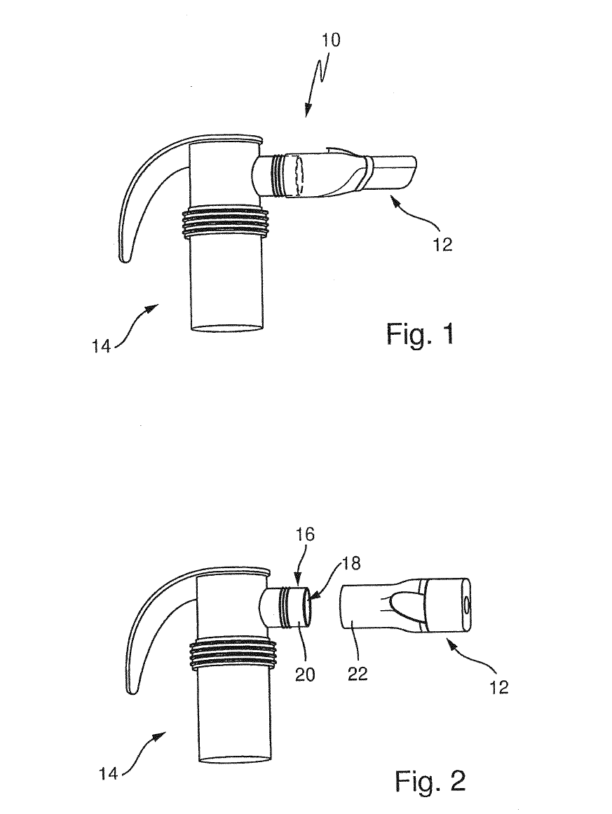

As can be seen from FIG. 2, the inhalation device 10 can be disassembled, in particular for cleaning purposes. The mouthpiece 12 can be pulled off the inhalation mixture generator 14.

The inhalation mixture generator 14 has a stub 16 onto which the mouthpiece 12 can be fitted releasably. The stub 16 is designed as a hollow tube section and surrounds an inhalation channel 18 through which the supplied inhalation mixture flows.

To be able to fit the mouthpiece 12 onto the stub 16, the stub 16 has a first plug section 20, which is designed to be joined to an associated and correspondingly designed second plug section 22 of the mouthpiece 12.

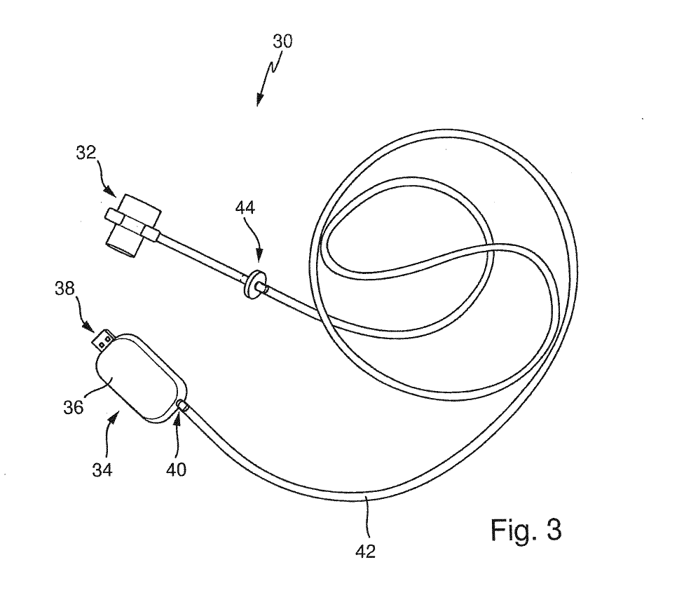

FIG. 3 shows an inhalation support apparatus 30 according to the invention that can be used with the inhalation device shown in FIGS. 1 and 2. The inhalation support apparatus has a pressure sensor adapter 32 and a control device 34.

The control device 34 has a housing 36 enclosing its electronic components. The housing 34 has a plug connector 38 which protrudes from the housing 34 and is rigidly connected to the housing 34 for producing a communication link with a data processing installation (not shown). The plug connector 38 is designed as a USB plug for plugging into a USB socket of the data processing installation.

The housing 36 of the control device 34 also has a pressure port 40. A flexible pressure sensor line 42 is attached to the pressure port 40 and serves to forward a pressure arising therein or pressure changes acting therein. The control device 34 comprises a pressure transducer (not shown) by which the pressure changes fed via the pressure sensor line 42 are converted into electrical measurement signals.

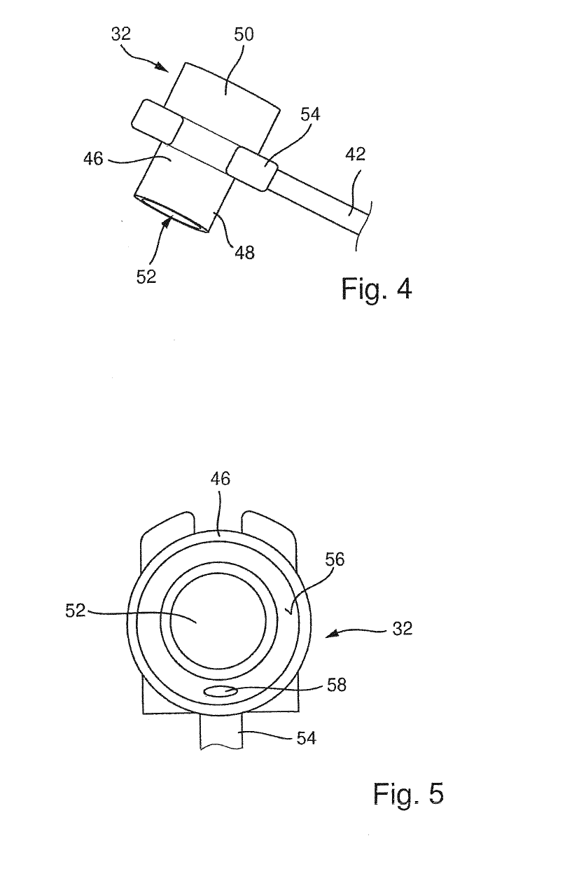

The other end of the pressure sensor line 42 is pressure-connected to the pressure sensor adapter 32 in a manner explained in more detail with reference to FIGS. 4 and 5, such that a pressure change can be detected by means of the pressure sensor adapter 32 and can be conveyed via the pressure sensor line 42 to the control device 34.

A filter element 44 is arranged in the pressure sensor line 42. This filter element 44 has a filter capsule, which encloses a filter material. The filter capsule has an inlet and an outlet, in such a way that a gaseous medium can flow through the inlet to the filter medium and, after passing through the filter medium, can flow out from the outlet. The filter medium is in particular designed in such a way that, during the passage of a gaseous medium with liquid droplets dissolved in the manner of an aerosol, the liquid droplets remain in the filter medium, and it is basically only the gaseous constituents that pass through.

FIG. 4 shows a detailed view of the pressure sensor adapter 32. The pressure sensor adapter 32 has a main body 46, which is designed in the manner of a hollow cylinder and which has an inlet plug section 48 and an outlet plug section 50. By means of the inlet plug section 48, the pressure sensor adapter 32 can be plugged onto the plug section 20 of the inhalation mixture generator 14. By way of the outlet plug section 50, it is possible to join the pressure sensor adapter to the corresponding plug section 22 of the mouthpiece 12. The resulting plug connection is releasable and can be easily disassembled again, for example for cleaning purposes.

The hollow cylindrical main body 46 surrounds a through-flow channel 52, which extends axially in the main body 46 and which is shown in more detail in FIG. 5. When the pressure sensor adapter 32 is connected, as has been explained, to the inhalation mixture generator 14 and to the mouthpiece 12, the inhalation mixture can flow from the inhalation mixture generator 14 through the inhalation channel 18 and via the through-flow channel 52 to the mouthpiece 12.

The main body 46 also has a pressure sensor port 54, which opens into the through-flow channel 52 through walls of the hollow cylindrical main body 46.

In FIG. 5, the pressure sensor adapter 32 is shown in a view looking into the through-flow channel 52 extending axially in the main body 46. A mouth 58 of the pressure sensor port 54 is indicated in the visible wall 56 of the main body 46. It will be seen that the through-flow channel 52 has a through-flow cross section much larger than the mouth cross section (pressure sensor cross section) of the mouth 58 of the pressure sensor port 54 (area ratio circa 10:1).

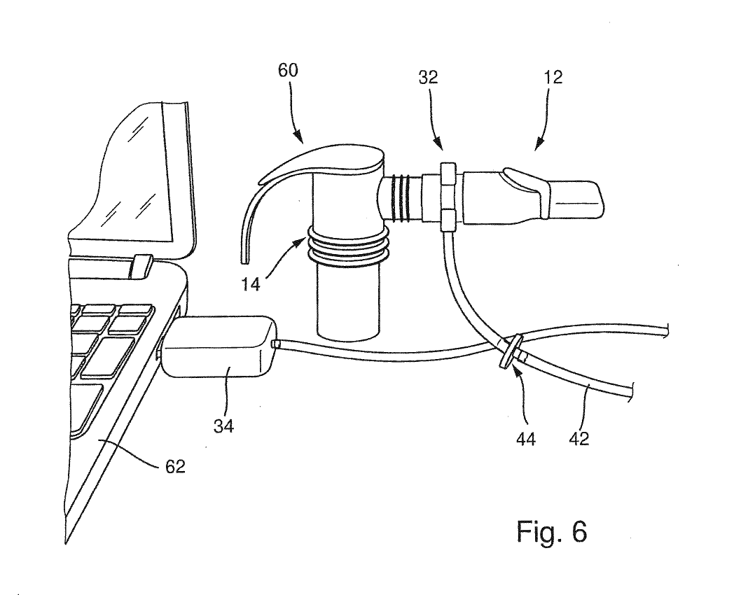

FIG. 6 shows an inhalation device 60 in which the pressure sensor adapter 32 of the inhalation support apparatus 30 shown in FIG. 3 is fitted between inhalation mixture generator 14 and mouthpiece 12.

The pressure sensor adapter 32 is pressure-connected to the control device 34 via the pressure sensor line 42. The pressure changes that occur in the inhalation channel (cf. reference sign 18 in FIG. 2) and/or in the through-flow channel (reference sign 52 in FIGS. 4 and 5) during an inhalation process can thus be forwarded from the pressure sensor adapter 32 to the control device 34 or can be fed thereto.

With the plug connector 38 explained with reference to FIG. 3, the control device 34 is attached to a corresponding socket (not shown) of a data processing installation (here a notebook PC) in order to establish a communication link between the control device 34 and the data processing installation 62.

A computer game, for example, runs on the data processing installation 62, the program code being stored in the control device 34 and being able to be transmitted to the data processing installation 62 via said communication link. This computer game can be controlled as a function of the pressure changes of the inhalation mixture (in the through-flow channel 52) that are detected by means of the pressure sensor adapter 32. For this purpose, the detected pressure changes can be fed via the pressure sensor line 42 to the control device 34 (via the pressure sensor port 54, cf. FIGS. 4 and 5). The control device 34 has a pressure transducer (not shown), by means of which the fed pressure changes can be converted into electrical measurement signals. From the electrical measurement signals, control signals for controlling the computer game running on the data processing installation 62 can be generated in the control device 34.

However, by means of a suitable computer program stored on the data processing installation 62, the electrical measurement signals generated in the control device 34 can also be used to generate measurement data providing information on the inhalation process. For example, parameters such as duration of inhalation, respiratory frequency, possible interruptions or pauses in breathing can be recorded, and these parameters can provide information on the course of the inhalation. With the aid of the measurement data thus obtained, the inhalation process can be further optimized on an individual basis for the patient in question.

### 2. Apparatus for Monitoring Pressure Changes

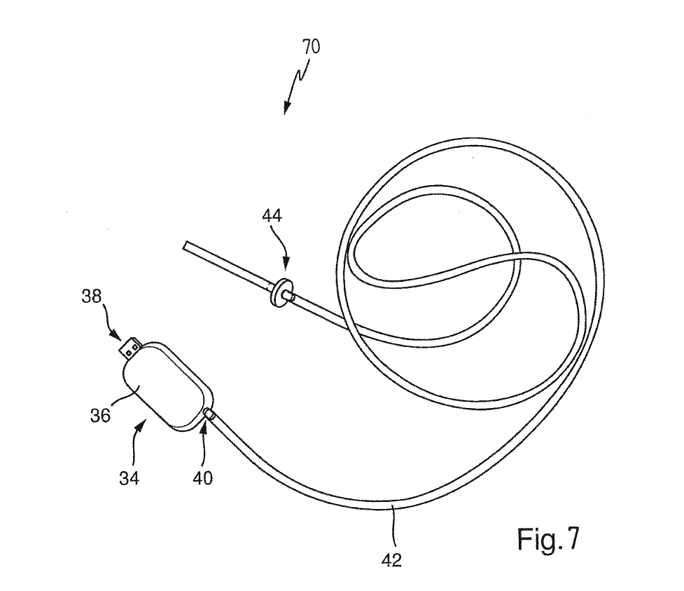

The control device 34 has a pressure transducer, as has been described above, and is in principle suitable for converting the pressure changes, fed via a pressure sensor line 42, into electrical measurement signals. A further embodiment of the invention thus concerns an apparatus 70 according to FIG. 7. This apparatus corresponds in principle to the apparatus 30 as shown in FIG. 3, but without the pressure sensor adapter 32 designed for use in inhalation equipment. This apparatus 70 preferably comprises a control device 34, which has a pressure transducer, a pressure sensor hose and, optionally, a filter element 44, wherein the control device 34 has a housing 36 with a pressure port 40. The control device 34 can optionally have a plug connector 38. This is especially the case when the connection to a data processing installation is produced not by a wireless connection but instead by a plug-type connection.

An apparatus set up in this way can be used wherever pressure changes occur and are able to be detected. The apparatus can be used both in medical and also non-medical fields.

An appliance on which the pressure changes are intended to be measured preferably has an attachment feature for the pressure sensor hose 42, or an attachment feature for the pressure sensor hose 42 can be retrofitted, such that the control device 34 is pressure-connected to the appliance for feeding the detected pressure changes to the control device 34.

By means of a suitable computer program stored on the data processing installation 62 or in the control device 34 itself, the electrical measurement signals generated in the control device 34 can be used to generate measurement data that can be used for the diagnosis of respiratory problems.

A method according to the invention for identifying respiratory problems comprises the following steps for example:
attaching the apparatus 70 to a data processing installation;
attaching the apparatus 70, by means of the pressure sensor hose 42, to an appliance for monitoring the respiration of a patient, in such a way that the control device 34 is pressure-connected to the appliance for monitoring the respiration;
detecting pressure changes and generating electrical measurement signals in the control device 34;
converting the electrical measurement signals from the control device 34 into measurement data by means of a suitable computer program stored on the data processing installation 62;
storing the measurement data on the data processing installation;
evaluating the measurement data, in particular comparing the measurement data obtained from a patient against the data from a healthy subject who has normal respiration;
determining divergences or impairments of the respiration of the patient in relation to the normal respiration of a healthy subject;
diagnosing the nature of the respiratory problems.

1: An inhalation support apparatus (30) for an inhalation device (10) that has a mouthpiece (12) and an inhalation mixture generator (14) for supplying an inhalation mixture to be inhaled,
comprising
a pressure sensor adapter (32), between the inhalation mixture generator (14) and the mouthpiece (12), detecting
pressure changes of the inhalation mixture;
and a control device (34) for controlling a program running on a data processing installation (62), wherein the control device (34) is configured to control the program as a function of the pressure changes.

2: The apparatus (30) as claimed in claim 1, wherein the control device (34) is pressure-connected to the pressure sensor adapter (32) feeding the detected pressure changes to the control device (34).

3: The apparatus (30) as claimed in claim 1, wherein the pressure sensor adapter (32) is releasably situated between the inhalation mixture generator (14) and the mouthpiece (12).

4: The apparatus (30) as claimed in claim 1, wherein the pressure sensor adapter (32) defines a through-flow channel (52) and a pressure sensor port (54) communicating pressure in a through-flow channel (52) to the control device (34).

5: The apparatus (30) as claimed in claim 1, wherein the pressure sensor adapter (32) is pressure-connected to the control device (34) via a flexible pressure sensor line (42) forwarding the detected pressure changes.

6: The apparatus (30) as claimed in claim 5, wherein a filter element (44) is provided in the pressure sensor line (42).

7: The apparatus (30) as claimed in claim 1, wherein the pressure sensor adapter (32) is free from electronic components, current-carrying components or voltage-carrying components.

8: The apparatus (30) as claimed in claim 1, wherein the control device (34) has a housing (36) provided with a plug connector (38) establishing a communication link with the data processing installation (62).

9: The apparatus (30) as claimed in claim 1, wherein the program is storable in the control device (34).

10-11. (canceled)

12: A method for treatment and/or alleviation of diseases, wherein the apparatus as claimed in claim 1 is used.

13-17. (canceled)

18: A method for supporting the inhalation of an inhalation mixture that is to be inhaled by means of an inhalation apparatus (60), said method comprising the following steps:
detecting a pressure change of the inhalation mixture that is to be inhaled;
converting the pressure change into control signals for controlling a data processing installation (62);
controlling a program running on the data processing installation (62) by the control signals and as a function of the pressure change.

19: (canceled)

20: An apparatus (70) for detecting pressure changes,
comprising
a control device (34),
a pressure sensor hose (42) operably connected to the control device (34), and
optionally a filter element (44) in the pressure sensor hose (42),
wherein the control device (34) is responsive to inhalation mixture pressure changes, has a housing (36) defining a pressure port (40) in communication with the pressure sensor hose (42), and, optionally, a communication link plug connector (38).

21: The apparatus (70) as claimed in claim 20 wherein, the control device (34) is pressure-connected to an apparatus in which pressure changes occur.

22. (canceled)

23: A method for diagnosis of respiratory problems comprising the use of the apparatus (70) as claimed in claim 20.

24. (canceled)

25: The method of claim 23, wherein said method is for the diagnosis of respiratory problems in sleep medicine.

26-28. (canceled)

29: The method of claim 23, wherein the apparatus (70) is used to monitor pressure changes.

30: The method as claimed in claim 29, comprising the steps of:
connecting the apparatus (70) to a data processing installation;
operably attaching the apparatus (70), by means of the pressure sensor hose (42), to an appliance for monitoring respiration of a patient, in such a way that the control device (34) is pressure-connected to the appliance for monitoring the respiration;
detecting pressure changes and generating electrical measurement signals in the control device (34);
converting the electrical measurement signals from the control device (34) into measurement data by means of a computer program stored on the data processing installation (62);
storing the measurement data on the data processing installation;
evaluating the measurement data, in particular comparing the measurement data obtained from a patient against the data from a healthy subject who has normal respiration;
determining divergences or impairments of the respiration of the patient in relation to the normal respiration of a healthy subject;
diagnosing the nature of the respiratory problems.

31: A process for monitoring respiration comprising the use of the apparatus (70) as claim in claim 20.

32-33. (canceled)

34: An inhalation support apparatus for an inhalation device which comprises
an inhalation mixture generator defining an inhalation channel;
a removable mouthpiece connected to the inhalation mixture generator and in communication with said inhalation channel;
a removable pressure sensor adapter between said mouthpiece and said inhalation mixture generator; and
a control device operably connected to said pressure sensor adapter and responsive to pressure changes in said inhalation channel.
